(19) United States
(12) Patent Application Publication
(10) Pub. No.: US 2002/0028255 A1
(43) Pub. Date: Mar. 7, 2002

(54) Pesticide treatment

(76) Inventors: Rachel Colegate, Ongar (GB); Mary C. Wells-Knecht, Wake Forest, NC (US); Anibal Lopes, Raleigh, NC (US)

Correspondence Address: Mary Katherine Baumeister, BURNS, DOANE, SWECKER & MATHIS, L.L.P., P.O. Box 1404, Alexandria, VA, 22313-1404, US

(21) Appl. No.: 09/855,607

(22) Filed: May 16, 2001

Related U.S. Application Data
(63) Continuation of application No. PCT/EP99/09254, filed on Nov. 16, 1999.
(60) Provisional application No. 60/108,603, filed on Nov. 16, 1998.

(51) Int. Cl. A62D 3/00; A61K 33/14

(52) U.S. Cl. 424/661; 588/205

(57) ABSTRACT

A method of accelerating the decomposition of an isoxazole and/or a dione pesticide by contacting them with halogen and/hypohalite. A crop seed containing a halogen and/or hypohalite within the seed or at its surface or in a coating on the seed.

### CROSS-REFERENCE TO RELATED APPLICATIONS

This is a continuation of International Application No. PCT/EP99/09254, filed Nov. 16, 1999, designating the United States, which International Application was published by the International Bureau on May 25, 2000, in English, as WO 00/29070. Priority of the above-mentioned International Application and of U.S. Provisional Application No. 60/108,603, filed Nov. 16, 1998, is hereby claimed. Each of the above-mentioned applications are hereby incorporated by reference in their entirety.

This invention relates to a method for the treatment of pesticides, e.g. herbicides, and their residues, to accelerate their decomposition.

Many isoxazole and dione pesticides, e.g. herbicides, are known. Following the application of such compounds, for example to control the growth of weeds, the isoxazoles may decompose to diones, in particular to diketonitrile compounds. This conversion is generally irreversible. The diketonitrile compounds, which are generally also pesticides, e.g. herbicides, may then decompose to the corresponding benzoic acids.

Despite the care taken to avoid contamination of the environment following the application of isoxazole herbicides or dione herbicides there remains a risk that small quantities may survive long enough to contaminate water supplies. In addition, there is always the need to clean the equipment used to apply such pesticides. The cleaning of the apparatus or mechanical devices or treatment of spillages is always difficult because the classical way of flushing out or chemical destruction generally more or less extend the contamination or pollution.

The present invention seeks to provide a method of accelerating the decomposition of isoxazole and/or dione pesticides. The invention also seeks to provide a method of cleaning equipment which has been in contact with such pesticides. The invention also seeks to provide a method of treating toxic spills of such herbicides in order to reduce the risk of wider environmental contamination. The invention also seeks to provide a method of protecting crop seeds from the pesticidal action of dione pesticides, e.g. herbicides.

An object of the instant invention is to provide a safe and efficient method of cleaning apparatus or mechanical devices which have been contaminated or polluted.

Another object of the invention is to provide a method of cleaning where there has been a contamination by pesticides, especially by pesticidally active diones.

Another object of the present invention is to provide a method of cleaning which does not create contaminated waste.

Other objectives will appear in the following description.

The above-mentioned aims and objects can be achieved, in whole or in part, by means of the present invention.

The present invention accordingly provides a method of accelerating the decomposition of an isoxazole pesticide and/or a dione pesticide by contacting them with halogen, e.g. chlorine and/or hypohalite, e.g. hypochlorite.

In this specification unless otherwise specified “halogen” refers to chlorine, bromine or iodine; “hypohalite” refers to hypochlorite, hypobromite or hypoiodite.

The halogen is preferably chlorine. The hypohalite is preferably hypochlorite. The halogen and/or hypohalite are generally used in an aqueous liquid.

When chlorine gas is dissolved in water there is an equilibrium with hypochlorite which may be represented by the following equilibria;

Cl₂+H₂O⇄HOCl+H⁺+Cl⁻

HOCl⇄H⁺+OCl⁻

The chlorine and/or hypochlorite used in the method of the invention can be provided in various ways. Aqueous chlorine can be dissolved in water. Hypochlorite is then formed by equilibration.

Alternatively a source of chlorine may be used. Such sources include hypochlorites and N-chloro compounds. The N-chloro compounds are related to chlorine by the equilibrium;

RR′NCl+H₂O⇄HOCl+RR′NH

where the group RR′N is a moiety known in the art. Similar N-bromo compounds yield hypobromite by hydrolysis.

The relative amounts of chlorine and hypochlorite present in an aqueous medium depend, inter alia, on the pH of the aqueous medium. Increasing the pH generally reduces the amount of available chlorine and increases the amount of hypochlorite. Reducing the pH generally increases the amount of available chlorine and reduces the amount of hypochlorite.

The hypochlorites are generally in the form of salts with alkali or alkaline earth metal salts. Sodium hypochlorite is preferred. The pH of sodium hypochlorite solutions for sale or storage is generally adjusted above pH 11 to maximize stability. Such solutions are available in various strengths, for example 5-6% sodium hypochlorite for household purposes or 10-15% for industrial purposes. Calcium hypochlorite is available in solutions containing, for example, 6-12% water and 65% available chlorine. In this specification, including the accompanying claims, percentages are by weight unless otherwise specified.

Bleaching powder and so-called tropical bleach may also be used as a solid source of chlorine. Bleaching powder is an indefinite, complex mixture of calcium hypochlorite, calcium hydroxide, calcium chloride and their hydrates. The amount of available chlorine varies generally from 24% to 37%. Its stability can be improved by adding calcium oxide. Such mixtures are known as tropical bleach as their stability is advantageous in hot, humid climates which would otherwise cause rapid decomposition. Such compounds have been used in the past to sanitize fields, drainage ditches and reservoirs where the insoluble material which they contain is of little importance. Other hypochlorites include dibasic magnesium hypochlorite and lithium hypochlorite.

N-chloro compounds include chlorinated isocyanurates, for example sodium dichloroisocyanurate dihydrate with about 56% chlorine. Anhydrous sodium and potassium salts of this compound are also available. Trichloroisocyanuric acid with 90% available chlorine may also be used for water treatment. Halogenated, e.g. chlorinated, hydantoins are stable solids which act as bleaches.

The isoxazole or dione pesticides treated by the method of the invention generally have the formula (I) (it is to be understood that the term “dione” as used in this specification including the accompanying claims does not exclude the possible presence of additional C═O groups as in triones):

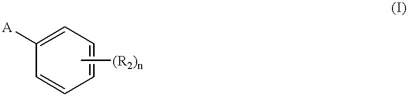

wherein:

A represents a group (A-1) to (A-7):

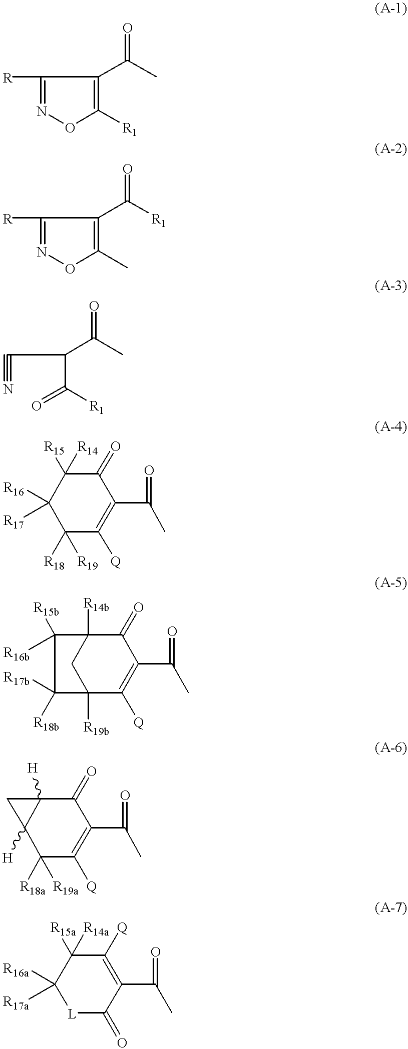

or a corresponding formula (A-6a) or (A-7a):

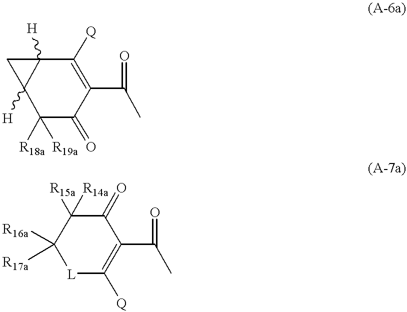

in which the position of the carbonyl group and the group Q are reversed and the double bond in the ring is attached to the carbon atom attached to the group Q;

R represents a hydrogen atom or a halogen atom; a straight- or branched chain alkyl, alkenyl or alkynyl group containing from one to six carbon atoms which is optionally substituted by one or more halogen atoms; a cycloalkyl group containing from 3 to 6 carbon atoms optionally substituted by one or more groups R⁵, one or more halogen atoms or a group —CO₂R³; or a group selected from —CO₂R³, —COR⁵, cyano, nitro, —CONR³R⁴ and —S(O)ₖR¹³;

R¹ represents a straight- or branched-chain alkyl, alkenyl or alkynyl group containing up to six carbon atoms which is optionally substituted by one or more halogen atoms; or a cycloalkyl group containing from three to six carbon atoms optionally substituted by one or more groups R⁵ or one or more halogen atoms;

R² represents a halogen atom; a straight- or branched-chain alkyl, alkenyl or alkynyl group containing up to six carbon atoms which is optionally substituted by one or more halogen atoms; a straight- or branched-chain alkyl group containing up to six carbon atoms which is substituted by one or more groups —OR⁵; or a group selected from nitro, cyano, —CO₂R⁵, —S(O)ₚR⁶, —O(CH₂)ₘOR⁵, —COR⁵, —NR¹¹R¹², —N(R⁸)SO₂R⁷, —N(R⁸)CO₂R⁷, —OR⁵, —OSO₂R⁷, —SO₂NR³R⁴, —CONR³R⁴, —CSNR³R⁴, —(CR⁹R¹⁰)ₜ—S(O)_{q}R⁷ and —SF₅;

or two groups R², on adjacent carbon atoms of the phenyl ring may, together with the carbon atoms to which they are attached, form a 5 to 7 membered saturated or unsaturated heterocyclic ring containing up to three ring heteroatoms selected from nitrogen, oxygen and sulfur, which ring is optionally substituted by one or more groups selected from halogen, nitro, —S(O)ₚR¹³, C₁₋₄ alkyl, C₁₋₄ alkoxy, C₁₋₄ haloalkyl, C₁₋₄ haloalkoxy, ═O (or a 5- or 6-membered cyclic acetal thereof), and ═NO—R³, it being understood that a sulphur atom, where present in the ring, may be in the form of a group —SO— or —SO₂—;

n represents an integer from one to five: when n is greater than one, the groups R² may be the same or different;

R³, R⁴ and R²² each independently represent a hydrogen atom, or a straight- or branched chain alkyl group containing up to six carbon atoms which is optionally substituted by one or more halogen atoms;

R⁵ and R²³ each independently represent a straight- or branched-chain alkyl group containing up to six carbon atoms which is optionally substituted by one or more halogen atoms or a straight- or branched-chain alkenyl or alkynyl group containing from two to six (preferably from three to six) carbon atoms which is optionally substituted by one or more halogen atoms;

R⁶ and R⁷, which may be the same or different, each represent R⁵; or phenyl optionally substituted by from one to five groups which may be the same or different, selected from a halogen atom, a straight- or branched-chain alkyl group containing up to six carbon atoms which is optionally substituted by one or more halogen atoms, nitro, cyano, —CO₂R⁵, —S(O)ₚR¹³, —NR¹¹NR¹², —OR⁵ and —CONR³R⁴;

R⁸, R⁹ and R¹⁰ each represent a hydrogen atom or R⁶;

R¹¹ and R¹² each represent hydrogen or R⁵;

R¹³ and R²¹ represent a straight- or branched-chain alkyl group containing up to six carbon atoms which is optionally substituted by one or more halogen atoms;

Q represents hydroxy, C₁₋₆ alkoxy, OR²⁰, SR²⁰ or SR²¹;

L represents oxygen or NR²²;

R¹⁴, R^{14a}, R_{14b}, R¹⁵, R^{15a}, R^{15b}, R¹⁶, R^{16a}, R^{16b}, R¹⁷, R^{17a}, R^{17b}, R¹⁸, R^{18a}, R^{18b}, R¹⁹, R^{19a} and R^{19b} represent the same or different groups selected from hydrogen, R²³, —(CH₂)ᵤCO₂R²², halogen, cyano, C₁₋₆ alkoxy, —(CH₂)ₓ-[phenyl optionally substituted by from one to five groups R²⁴ which may be the same or different], and cycloalkyl containing from three to six carbon atoms optionally substituted by C₁₋₆ alkyl or —S(O)ₚR²¹;

R²⁰ represents phenyl optionally substituted by from one to five groups selected from halogen, C₁₋₆ alkyl, C₁₋₆ haloalkyl, C₁₋₆ alkoxy and nitro;

R²⁴ represents a group selected from halogen, R²⁵, nitro, cyano, —CO₂R²⁶, —S(O)ₚR²¹, —OR²¹ and —NR²⁶R²⁷;

R²⁵ represents a straight- or branched- chain alkyl group containing one to three carbon atoms optionally substituted by one or more halogen atoms;

R²⁶ and R²⁷ which may be the same or different, each represents hydrogen or R²³;

p, q and u independently represent the values zero, one or two;

k and m represents one, two or three;

x represents zero or one;

t represents an integer from one to four; when t is greater than one, the groups R⁹ and R¹⁰ may be the same or different;

and agriculturally acceptable salts and metal complexes thereof.

Compounds of formula (I) may exist in enolic tautomeric forms that may give rise to geometric isomers around the enolic double bond. Furthermore in certain cases the above substituents may contribute to optical isomerism and/or stereoisomerism. All such forms and mixtures are embraced by the present invention.

It is to be understood that in this specification compounds comprising a cyclohexane ring corresponding to formula (A-6) or (A-7) or a precursor thereof include the compounds with the corresponding formula (A-6a) or (A-7a) or precursors thereof.

In the definitions of symbols in this specification including the accompanying claims unless otherwise specified the following term is generally defined thus:-

‘halogen’ means a fluorine, chlorine, bromine or iodine atom.

Preferably R¹ represents a group of formula (A-1), (A-2), (A-3) or (A-4) (compounds of formula (A-1) are most preferred).

The benzoyl ring of the compounds of formula (I) is preferably 2,4-disubstituted, 2,3-disubstituted or 2,3,4-trisubstituted.

Preferably in formulae (A-4) to (A-7), the groups R¹⁴, R¹⁵, R¹⁶, R¹⁷, R¹⁸, R¹⁹, R^{14a}, R^{15a}, R^{16a}, R^{17a}, R^{14b}, R^{15b}, R^{16b}, R^{17b}, R^{18b} and R^{19b} represent hydrogen or lower alkyl (preferably hydrogen, methyl or ethyl); L (in A-7a) represents NH; and Q represents hydroxy or —S-phenyl.

Compounds of formula (I) in which A represents (A-1), (A-2) and (A-3); R represents hydrogen or —CO₂R₃ (in A-1 or A-2) wherein R³ represents a straight- or branched chain alkyl group containing up to three carbon atoms; and R¹ represents cyclopropyl are preferred.

A further preferred class of compounds of formula (I) wherein A represents (A-1) are those wherein:

R is hydrogen or —CO₂Et;

R¹ is cyclopropyl;

and two groups R², on adjacent carbon atoms of the phenyl ring may, together with the carbon atoms to which they are attached, combine to form a 5 or 6 membered saturated or unsaturated heterocyclic ring which is fused to the 2,3 or 3,4 positions of the benzoyl ring; wherein the heterocyclic ring contains two hetero atoms selected from sulphur and oxygen which are located at the 2 and 3, or 3 and 4 positions of the benzoyl ring; and in which the 4-substituent of the benzoyl ring is halogen or S(O)ₚMe, or the 2-substituent of the benzoyl ring is methyl, S(O)ₚMe or —CH₂S(O)_{q}Me respectively; and optionally the heterocyclic ring may be substituted by one or more halogen atoms.

A further preferred class of compounds of formula (I) are those wherein A represents (A-1); R is hydrogen or —CO₂Et; R¹ is cyclopropyl; R² is a halogen atom or a group selected from —CF₃, Me, Et, —S(O)ₚMe, —CH₂S(O)_{q}Me and optionally halogenated methoxy or ethoxy; and n is two or three.

A further preferred class of compounds of formula (I) wherein A represents (A-4) are those wherein:

R¹⁴, R¹⁵, R¹⁶, R¹⁷, R¹⁸ and R¹⁹ represent hydrogen;

and two groups R², on adjacent carbon atoms of the phenyl ring may, together with the carbon atoms to which they are attached, combine to form a 6 membered saturated heterocyclic ring which is fused to the 2,3 or 3,4 positions of the benzoyl ring; wherein the heterocyclic ring contains a sulphur atom located at the 4 position of the benzoyl ring, optionally in the form of a group —SO— or —SO₂—, and which ring is substituted by a 5 or 6-membered cyclic acetal thereof.

A more preferred class of compounds of formula (I) having the formula (Ia):

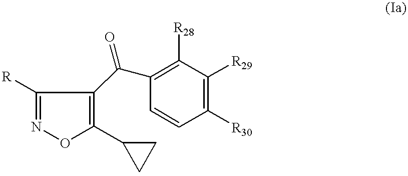

are those wherein:

R is hydrogen or —CO₂Et;

R²⁸ is selected from —S(O)ₚMe, Me, Et, a chlorine, bromine or fluorine atom, methoxy, ethoxy and —CH₂S(O)_{q}Me;

R²⁹ is selected from a hydrogen atom, a chlorine, bromine or fluorine atom, methoxy, ethoxy and —S(O)ₚMe; and

R³⁰ is selected from a hydrogen atom, a chlorine, bromine or fluorine atom, methoxy and CF₃.

An especially preferred class of compounds of formula (I) have the formula (Ib):

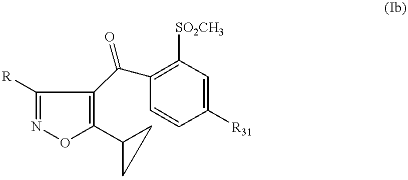

wherein R³¹ is chlorine, bromine or trifluoromethyl; and

R is hydrogen or —CO₂Et.

Preferred diones are those in which a substituted phenyl ring as defined in formula (I); (Ia); or (Ib), is attached to a grouping

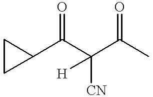

Such diones in which the phenyl ring is substituted by two groups independently selected from halogen, alkyl, S(O)ₜ alkyl (t=0, 1 or 2) or haloalkyl are also preferred. Alkyl groups and moieties in this specification, unless otherwise specified, are of 1 to 6 carbon atoms.

Preferred triones are those in which a substituted phenyl ring, as defined above, is attached to a grouping:

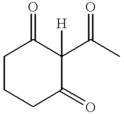

The following compounds of formula (I) are among the most preferred for use in the present invention:

5-cyclopropyl-4-[2-chloro-3-ethoxy-4-(ethylsulphonyl)benzoyl]isoxazole;

4-(4-chloro-2-methylsulphonylbenzoyl)-5-cyclopropylisoxazole;

5-cyclopropyl-4-(2-methylsulphonyl-4-trifluoromethylbenzoyl)isoxazole;

4-(4-bromo-2-methylsulphonylbenzoyl)-5-cyclopropylisoxazole;

5-cyclopropyl-4-[4-fluoro-3-methoxy-2-(methylsulphonyl)benzoyl]isoxazole;

4-(4-bromo-2-methylsulphonylmethylbenzoyl)-5-cyclopropylisoxazole;

ethyl 5-cyclopropyl-4-(2-methylsulphonyl-4-trifluoromethylbenzoyl)isoxazole-3-carboxylate;

2-[2-chloro-(4-methylsulphonyl)benzoyl]-1,3-cyclohexanedione;

2-[2-nitro-4-(methylsulphonyl)benzoyl]-1,3-cyclohexanedione;

2-(2,3-dihydro-5,8-dimethyl-1,1-dioxospiro[4H-1-benzothiin-4,2′-[1,3]dioxolan]-6-ylcarbonyl)cyclohexane-1,3-dione;

5-cyclopropyl-4-(2-methylsulphonyl-4-trifluoromethylbenzoyl)-3-methylthioisoxazole; and

2-cyano-3-cyclopropyl-1-(2-methylsulphonyl-4-trifluoromethylphenyl)propan-1,3-dione.

The most preferred compound is 5-cyclopropyl-4-(2-methylsulphonyl-4-trifluoromethylbenzoyl)isoxazole.

The material used as a source of chlorine in the method of the invention can be chosen by the normally skilled worker taking into account, for example, the location and concentration of the material to be treated.

Dione pesticides may be present at very low concentrations in ground water (for example at the ppb level, e.g. 1 ppb i.e. one part in 10⁹). The invention provides a method of treating water containing a low concentration of an isoxazole and/or dione pesticide, e.g. herbicide which comprises contacting the water with halogen e.g. chlorine, and/or hypohalite, e.g. hypochlorite. Chlorination of water to a concentration of 0.1 ppb or more, preferably 0.1 ppb to 0.5 ppm, more preferably to 0.3 ppm can be used in the method of the invention to treat such contamination with isoxazole and/or dione.

The invention also provides a method of cleaning a surface which is contaminated or believed to be contaminated by a dione and/or isoxazole pesticide, the said method comprising the application to the said surface of an effective amount of an aqueous liquid containing halogen, e.g. chlorine and/or hypohalite, e.g. hypochlorite.

The surfaces which are cleaned according to the invention may be outer or inner surfaces of containers, pipes, nozzles, sprayers etc.

The cleaning method of the invention is generally effected using an aqueous solution comprising 10⁻⁴ to 2 moles of chlorine per liter, preferably 0.01 to 0.5 moles. The pH of the aqueous cleaning solution may be in the range of 4 to 10, preferably 5 to 9, more preferably 7 to 9. The pH of commercially available solutions which may have a higher pH can, if desired, be adjusted to the desired value by known methods.

The application of the solution is generally done at ambient temperature. However, application of warm solution may also be possible.

The application is done with a duration of contact generally in the range of 10 minutes to 5 hours, preferably 20 minutes to 1 hour. The duration of such cleaning may vary considerably and these limits are not crucial, and can be determined easily by the person skilled in the art.

The solutions are preferably used on plastics materials. When used on metallic services care may be necessary to reduce the chance of corrosion, for example by limiting the contact time with the cleaning solution.

Cleaning solutions should have a pH higher than 7.5, preferably higher than 8 (and generally less than 11) if they are used on isoxazoles. The cleaning solutions described above may also be used to treat spills of isoxazole and/or dione herbicides.

The invention also provides a method for reducing the spread of isoxazole and/or dione pesticide from a first locus comprising the said pesticide to a second locus which method comprises applying to a third locus situated between the first locus and the second locus a halide and/or hypohalite. The halide and/or hypohalite are preferably applied, generally in aqueous solution, to the soil at the edge of an area comprising the dione and/or isoxazole pesticide to accelerate decomposition of the pesticide. It will be understood that the first locus may be an area treated with the dione or a precursor thereof, for example an isoxazole. This method of the invention seeks to reduce the spread of pesticide from a treated area. According to an alternative embodiment of the invention the second locus may be an area comprising plants which it is desired to protect from dione and/or isoxazole pesticide.

When transmission of pesticide is associated with movement of ground water the third locus may be a drainage ditch. Furthermore, where the direction of movement of ground water through an area comprising the dione and/or isoxazole pesticide is known the method of the invention may be used to treat water draining from the area. For example the method may comprise digging a drainage ditch generally across the direction of water movement. The ditch itself can then be treated with halogen and/or hypohalite.

The invention also provides a crop seed having increased resistance to herbicidal diones and/or isoxazoles, which seed comprises an effective amount of halogen, e.g. chlorine, and/or hypohalite, e.g. hypochlorite, either within the seed or at its surface or in a coating on the seed. Isoxazoles may act as precursors of the diones.

The amount of available halogen, e.g. chlorine, is preferably in the range of 10⁻⁵ to 1% (by weight) of the said seed, preferably 10⁻³ to 0.1%.

The main utility of the said seed is when sown in an area which is for cropping and from which weeds are going to be eliminated by means of a herbicidally effective amount of an active ingredient of formula I.

The seed can be prepared by a process which comprises contacting, e.g. by soaking or dipping or spraying the untreated seed with a solution of halogen, e.g. chlorine and/or hypohalite, e.g. hypochlorite. The seed may also be coated by a coating comprising the halogen and/or hypohalite. The coating may also contain all kinds of other ingredients according to the particular purpose of the said coating. Coatings and their contents are known in the literature and especially in U.S. Pat. No. 5,087,475, and in the prior art referred to in that patent, their disclosure being incorporated herein by reference.

The invention is illustrated by the following non-limiting Examples.

### EXAMPLES

### Example 1

(A) The kinetics of the decomposition of 2-cyano-3-cyclopropyl-1-(2-methylsulphonyl-4-trifluoromethylphenyl)propan-1,3-dione in water was studied. The following Table summarizes the results obtained at pH7 or pH9 in the presence of 0.1 or 0.5 ppm of chlorine. The percentages given are of degradation of the dione to the corresponding benzoic acid, 2-methylsulphonyl-4-trifluoromethyl-benzoic acid.

TABLE 1

| pH | Cl₂ (ppm) | 15 min | 60 min |
|---|---|---|---|
| 7 | 0.1 | 100% | |
| 7 | 0.5 | 100% | |
| 9 | 0.1 | 50% | 100% |
| 9 | 0.5 | 100% | |

(B) Vials containing 1 ml of solutions in water of the dione used in (A) (at a concentration of 500 ppt) were treated at ambient temperature with 1 ml of water containing 0.1 or 0.5 ppb concentrations of chlorine.

The following results were obtained.

| pH | Cl₂ (ppb) | 15 min | 60 min |
|---|---|---|---|
| 5 | 0.1 | 100% | |
| 5 | 0.5 | 100% | |
| 7 | 0.1 | 100% | |
| 7 | 0.5 | 100% | |
| 9 | 0.1 | | >90% |
| 9 | 0.5 | 100% | |

### Example 2

2-Cyano-3-cyclopropyl-1-2-methylsulphonyl-4-trifluoromethylphenyl)propan-1,3-dione(DKN:1 g) was added to 25 ml of bleach (containing 14 to 15% chlorine). The mixture was stirred at ambient temperature. After 3 minutes a clear solution formed. After 30 minutes no DKN was detected by thin layer chromatography (TLC). The main product was the corresponding benzoic acid.

### Example 3

A 1000 l container or tank was used to prepare a suspension of dione pesticide (2-cyano-3-cyclopropyl- 1-(2-methylsulphonyl-4-trifluoromethylphenyl)propan-1,3-dione). The suspension was used to destroy the weeds in a crop growing area. The container was then still wet with aqueous suspension of 0.02% (w/w) of the pesticide.

10 l of 0.01% (w/w) solution of sodium hypochlorite is poured into the tank and stirred (so as to contact the whole inner surface of the container) and left for one hour.

All of the dione pesticide disappeared.

### Example 4

Example 3 is repeated except that the pesticide is an isoxazole (5-cyclopropyl-4-(2-methylsulphonyl-4-trifluoromethylbenzoyl)isoxazole;IFT) and the pH of the hypochlorite solution is 8.5 (adjusted using NaOH). A similar result is obtained.

### Example 5

Maize (Pioneer 3394) seeds were placed in a solution of 1% (w/w) sodium hypochlorite for 30 seconds, then rinsed thoroughly for 5 minutes with sodium bicarbonate solution (5% w/w).

Replicates of 20 seeds from each treatment were placed in petri dishes and bathed in 25 ml of the following solutions for 24 hours (covered and incubated at 25° C. in a growth cabinet).

| | |
|---|---|
| Solution 1 | water (control) |
| Solution 2 | 2% Acetone (control) |
| Solution 3 | 0.1 μg ml⁻¹ IFT |
| Solution 4 | 1.0 μg ml⁻¹ IFT |
| Solution 5 | 10.0 μg ml⁻¹ IFT |
| Solution 6 | 1.0 μg ml⁻¹ DKN |
| Solution 7 | 10.0 μg ml⁻¹ DKN |
| Solution 8 | 100.0 μg ml⁻¹ DKN |

After 24 hours the seeds were removed from the bathing solutions, rinsed thoroughly in water and planted (in green light- as some of the seeds had started to produce a coleoptile which is sensitive to daylight). Seeds were planted in duplicate in 10 cm×10 cm pots containing non-sterile loam and were overhead watered 3 times daily.

Assessments were made 7 and 14 DAT (days after treatment).

### RESULTS

No damage was produced by low concentrations of either IFT (0.1 μg ml⁻¹) or DKN (1 μg ml⁻¹).

Sterilization of the seed coat increases the seed's resistance to herbicide uptake, i.e. the resultant activity in the shoot (evident by bleaching) is less than that of the control.

% Damage (bleaching of the shoot)

| | Control seed | Sterilized seed |
|---|---|---|
| Solution 1 | 0 | 0 |
| Solution 2 | 0 | 0 |
| Solution 3 | 0 | 0 |
| Solution 4 | 20 | 13 |
| Solution 5 | 98 | 100 |
| Solution 6 | 0 | 0 |
| Solution 7 | 0 | 0 |
| Solution 8 | 87 | 11 |

While the invention has been described in terms of various preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What we claim is:

1. A method of accelerating the decomposition of an isoxazole and/or a dione pesticide comprising contacting said pesticide with halogen and/or hypohalite.

2. A method according to claim 1 in which the halogen is chlorine and the hypohalite is hypochlorite.

3. A method according to claim 1 in which the halogen and/or hypohalite are used in an aqueous liquid.

4. A method according to claim 3 in which the hypohalite is a salt with an alkali or alkaline earth metal.

5. A method according to claim 1 in which the hypohalite is sodium hypochlorite.

6. A method according to claim 1 in which the isoxazole and/or dione are of the formula: 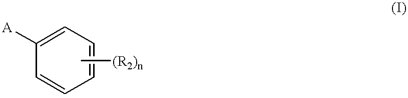
wherein:
A represents a group (A-1) to (A-7): 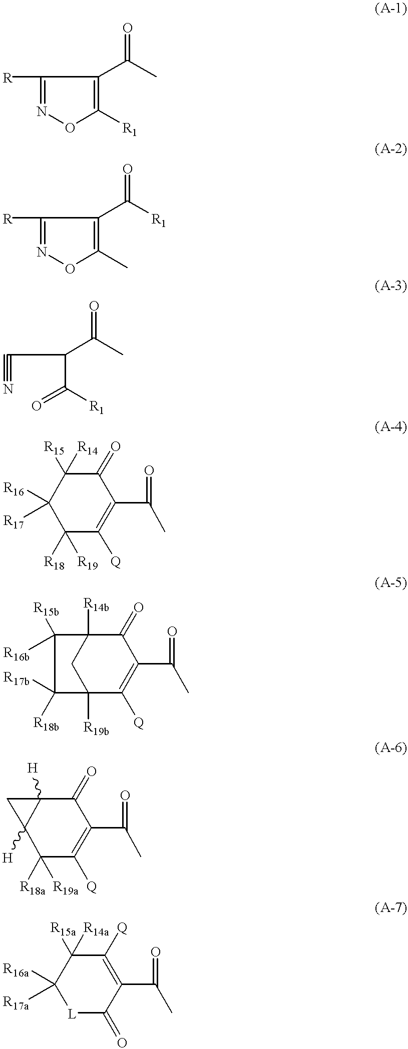
or a corresponding formula (A-6a) or (A-7a): 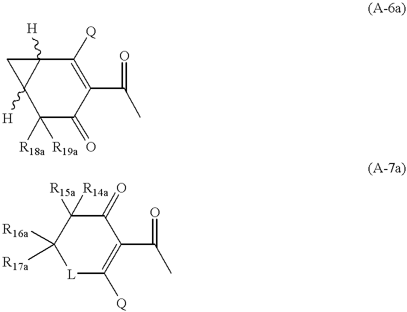
in which the position of the carbonyl group and the group Q are reversed and the double bond in the ring is attached to the carbon atom attached to the group Q;
R represents a hydrogen atom or a halogen atom; a straight- or branched chain alkyl, alkenyl or alkynyl group containing from one to six carbon atoms which is optionally substituted by one or more halogen atoms; a cycloalkyl group containing from 3 to 6 carbon atoms optionally substituted by one or more groups R⁵, one or more halogen atoms or a group —CO₂R³; or a group selected from —CO₂R³, —COR⁵, cyano, nitro, —CONR³R⁴ and —S(O)ₖR¹³;
R¹ represents a straight- or branched-chain alkyl, alkenyl or alkynyl group containing up to six carbon atoms which is optionally substituted by one or more halogen atoms; or a cycloalkyl group containing from three to six carbon atoms optionally substituted by one or more groups R⁵ or one or more halogen atoms;
R² represents a halogen atom; a straight- or branched-chain alkyl, alkenyl or alkynyl group containing up to six carbon atoms which is optionally substituted by one or more halogen atoms; a straight- or branched-chain alkyl group containing up to six carbon atoms which is substituted by one or more groups —OR⁵; or a group selected from nitro, cyano, —CO₂R⁵, —S(O)ₚR⁶, —O(CH₂)ₘOR⁵, —COR⁵, —NR¹¹R¹², —N(R⁸)SO₂R⁷, —N(R⁸)CO₂R⁷, —OR⁵, —OSO₂R⁷, —SO₂NR³R⁴, —CONR³R⁴, —CSNR³R⁴, —(CR⁹R¹⁰)ₜ—S(O)_{q}R⁷ and —SF₅;
or two groups R², on adjacent carbon atoms of the phenyl ring may, together with the carbon atoms to which they are attached, form a 5 to 7 membered saturated or unsaturated heterocyclic ring containing up to three ring heteroatoms selected from nitrogen, oxygen and sulfur, which ring is optionally substituted by one or more groups selected from halogen, nitro, —S(O)ₚR¹³, C₁₋₄ alkyl, C₁₋₄ alkoxy, C₁₋₄ haloalkyl, C₁₋₄ haloalkoxy, ═O (or a 5- or 6-membered cyclic acetal thereof), and ═NO—R³, it being understood that a sulphur atom, where present in the ring, may be in the form of a group —SO— or —SO₂—;
n represents an integer from one to five: when n is greater than one, the groups R² may be the same or different;
R³, R⁴ and R²² each independently represent a hydrogen atom, or a straight- or branched chain alkyl group containing up to six carbon atoms which is optionally substituted by one or more halogen atoms;
R⁵ and R²³ each independently represent a straight- or branched-chain alkyl group containing up to six carbon atoms which is optionally substituted by one or more halogen atoms or a straight- or branched-chain alkenyl or alkynyl group containing from two to six (preferably from three to six) carbon atoms which is optionally substituted by one or more halogen atoms;
R⁶ and R⁷, which may be the same or different, each represent R⁵; or phenyl optionally substituted by from one to five groups which may be the same or different selected from a halogen atom, a straight- or branched-chain alkyl group containing up to six carbon atoms which is optionally substituted by one or more halogen atoms, nitro, cyano, —CO₂R⁵, —S(O)ₚR¹³, —NR¹¹NR¹², —OR⁵ and —CONR³R⁴;
R⁸, R⁹ and R¹⁰ each represent a hydrogen atom or R⁶;
R¹¹ and R¹² each represent hydrogen or R⁵;
R¹³ and R²¹ represent a straight- or branched-chain alkyl group containing up to six carbon atoms which is optionally substituted by one or more halogen atoms;
Q represents hydroxy, C₁₋₆ alkoxy, OR²⁰, SR²⁰ or SR²¹;
L represents oxygen or NR²²;
R¹⁴, R^{14a}, R^{14b}, R¹⁵, R^{15a}, R^{15b}, R¹⁶, R^{16a}, R^{16b}, R¹⁷, R^{17a}, R^{17b}, R¹⁸, R^{18a}, R^{18b}, R¹⁹, R^{19a} and R^{19b} represent the same or different groups selected from hydrogen, R²³, —(CH₂)ᵤCO₂R²², halogen, cyano, C₁₋₆ alkoxy, —(CH₂)ₓ-[phenyl optionally substituted by from one to five groups R²⁴ which may be the same or different], and cycloalkyl containing from three to six carbon atoms optionally substituted by C₁₋₆ alkyl or —S(O)ₚR²¹;
R²⁰ represents phenyl optionally substituted by from one to five groups selected from halogen, C₁₋₆ alkyl, C₁₋₆ haloalkyl, C₁₋₆ alkoxy and nitro;
R²⁴ represents a group selected from halogen, R²⁵, nitro, cyano, —CO₂R²⁶, —S(O)ₚR²¹, —OR²¹ and —NR²⁶R²⁷;
R²⁵ represents a straight- or branched-chain alkyl group containing one to three carbon atoms optionally substituted by one or more halogen atoms;
R²⁶ and R²⁷ which may be the same or different, each represents hydrogen or R²³;
p, q and u independently represent the values zero, one or two;
k and m represent one, two or three;
x represents zero or one;
t represents an integer from one to four; when t is greater than one, the groups R⁹ and R¹⁰ may be the same or different;
or an agriculturally acceptable salt or metal complex thereof.

7. A method according to claim 1 for treating water containing a low concentration of an isoxazole and/or dione pesticide which comprises contacting the water with halogen and/or hypohalite.

8. A method according to claim 7 in which the water is chlorinated to a concentration of 0.1 ppb to 0.5 ppm.

9. A method according to claim 1 for cleaning a surface which is contaminated or believed to be contaminated by a dione and/or isoxazole pesticide which method comprises applying to said surface an effective amount of an aqueous liquid containing halogen and/or hypohalite.

10. A method according to claim 9 in which the aqueous liquid is an aqueous solution containing 10⁻⁴ to 2 moles of chlorine per liter.

11. A method according to claim 10 in which the pH of the cleaning solution is from 7 to 9.

12. A method according to claim 1 for reducing the spread of isoxazole and/or dione pesticide from a first locus to a second locus which comprises applying to a third locus situated between the first locus and the second locus a halide and/or a hypohalite.

13. A method according to claim 1 to confer on a crop seed increased resistance to a herbicidal isoxazole and/or dione which method comprises treating the seed with halogen and/or hypohalite or providing on the surface of the seed a coating comprising halogen and/or hypohalite.

14. A method according to claim 1 in which the isoxazole and/or dione is selected from the group consisting of:
5-cyclopropyl-4-[2-chloro-3-ethoxy-4-(ethylsulphonyl)benzoyl]isoxazole;
4-(4-chloro-2-methylsulphonylbenzoyl)-5-cyclopropylisoxazole;
5-cyclopropyl-4-(2-methylsulphonyl-4-trifluoromethylbenzoyl)isoxazole;
4-(4-bromo-2-methylsulphonylbenzoyl)-5-cyclopropylisoxazole;
5-cyclopropyl-4-[4-fluoro-3-methoxy-2-(methylsulphonyl)benzoyl]isoxazole;
4-(4-bromo-2-methylsulphonylmethylbenzoyl)-5-cyclopropylisoxazole;
ethyl 5-cyclopropyl-4-(2-methylsulphonyl-4-trifluoromethylbenzoyl)isoxazole-3-carboxylate;
2-[2-chloro-(4-methylsulphonyl)benzoyl]-1,3-cyclohexanedione;
2-[2-nitro-4-(methylsulphonyl)benzoyl]-1,3-cyclohexanedione;
2-(2,3-dihydro-5,8-dimethyl-1,1-dioxospiro[4H-1-benzothiin-4,2′-[1,3]dioxolan]-6-ylcarbonyl)cyclohexane-1,3-dione;
5-cyclopropyl-4-(2-methylsulphonyl-4-trifluoromethylbenzoyl)-3-methylthioisoxazole; and
2-cyano-3-cyclopropyl-1-(2-methylsulphonyl-4-trifluoromethylphenyl)propan-1,3-dione.

15. A method according to claim 14 in which the isoxazole and/or dione comprises 5-cyclopropyl-4-(2-methylsulphonyl-4-trifluoromethylbenzoyl)isoxazole.

16. A crop seed having increased resistance to a herbicidal isoxazole and/or dione, which seed comprises an effective amount of halogen and/or hypohalite either within the seed or at its surface or in a coating on the seed.

17. A seed according to claim 13 in which the amount of halogen is from 10⁻³ to 0.1% by weight.
